(19) United States
(12) Patent Application Publication
(10) Pub. No.: US 2023/0355094 A1
(43) Pub. Date: Nov. 9, 2023

(54) TRANSDUCER PROBES FOR OPHTHALMOLOGICAL INSTRUMENTS AND USES THEREOF

(71) Applicant: CATS TONOMETER, LLC, Tucson, AZ (US)

(72) Inventor: Sean J. McCafferty, Tucson, AZ (US)

(73) Assignee: CATS TONOMETER, LLC, Tucson, AZ (US)

(21) Appl. No.: 18/209,687

(22) Filed: Jun. 14, 2023

Related U.S. Application Data
(63) Continuation of application No. PCT/US2022/040617, filed on Aug. 17, 2022.
Application No. PCT/US2022/040617 is a continuation-in-part of application No. 17/889,713, filed on Aug. 17, 2022.
(60) Provisional application No. 63/252,472, filed on Oct. 5, 2021.
(60) Provisional application No. 63/252,472, filed on Oct. 5, 2021.
(60) Provisional application No. 63/242,752, filed on Sep. 10, 2021.
(60) Provisional application No. 63/242,752, filed on Sep. 10, 2021.
(60) Provisional application No. 63/235,058, filed on Aug. 19, 2021.
(60) Provisional application No. 63/235,058, filed on Aug. 19, 2021.
(60) Provisional application No. 63/235,026, filed on Aug. 19, 2021.
(60) Provisional application No. 63/235,026, filed on Aug. 19, 2021.
(60) Provisional application No. 63/235,024, filed on Aug. 19, 2021.
(60) Provisional application No. 63/235,024, filed on Aug. 19, 2021.

(51) Int. Cl. A61B 3/16

(52) U.S. Cl. CPC A61B 3/16

(57) ABSTRACT

Transducer probes for ophthalmological instruments operating without the use of light (such as contact tonometers and ultrasound pachymeters) - whether structured substantially monolithically (as one piece) or in a hybrid or composite fashion (and, therefore, reversibly separable into constituent components) - the axially-located front transducer surface of which is surrounded by a ridge of material not representing the surface of the transducer, in which the overall front comea-contacting surface of which is inwardly shaped to reduce or even completely avoid measurement errors associated with the use of conventionally-structured versions of probes traditionally use with such instruments. Ophthalmological instruments utilizing such probes and methods of use. Biological protection of eye during use of such instruments.

### CROSS-REFERENCE TO RELATED APPLICATIONS

This U.S. Pat. Application is a continuation of the International Patent Application PCT/US2022/040617, which claims priority from and benefit of the U.S. Provisional Pat. Applications Nos. 63/235,058 filed on Aug. 19, 2021; 63/235,024 filed on Aug. 19, 2021; 63/235,026 filed on Aug. 19, 2021; 63/242,752 filed on Sep. 10, 2021; and 63/252,472 filed on Oct. 05, 2021, as well as from the U.S. Pat. Application No. 17/889,713 filed on Aug. 17, 2022 and now published as US 2023/0056896. The disclosure of each of the above-identified patent documents is incorporated by reference herein.

### TECHNICAL FIELD

The present invention relates generally to the field of ophthalmological instruments (such as contact tonometers or ultrasound pachymeters) equipped with and employing a transducer for registration of the target measurement data and, more specifically, to such ophthalmological instruments employing a transducer probe with a spatially-curved eye-contacting surface.

### RELATED ART

Multiple reliable instruments are being currently used in ophthalmology including, to name just a few, a tonometer (used for testing intraocular pressure) and a pachymeter (used for measurement of thickness of the cornea prior to refractive surgery, for example), both of which instruments are advantageous in screening for patients suspected of developing glaucoma, for example. Glaucoma is characterized by increase in pressure within the eye, but because the patient seldom experiences any symptoms until major damage occurs, regular testing is essential to detect glaucoma in the early state before the retinal field is seriously diminished, and ocular nerve damage has occurred.

Utilizing “contact” types of such devices - especially those that are hand-held - requires at least touching the eye surface directly with the probe of a device and, in some cases, pressing the tip against the cornea into the eye thereby indenting a portion of the cornea. (In this context, the probe of the contact instrument is understood to be that protruding part of it which is juxtaposed and/or cooperated with an eye under test. (In some of related art documents, such probe portion may also and/or interchangeably be referred to as a “tip”.) Practice shows that the use of conventionally-structured transducer-containing ophthalmological instrument - while often preferred because such use does not require any specific technical preparation and/or education of the user to assess the results of the measurements - almost always produces the values of targeted measured parameter (be it the IOP or a corneal thickness) that are substantially lower than the those identified with the use of more accurate and complex instruments (in the case of measurement of the IOP, for example - a Goldmann-type tonometer) which admittedly require much more expertise and clinical time to measure.

### BRIEF DESCRIPTION OF THE DRAWINGS

The invention will be more fully understood by referring to the following Detailed Description of Specific Embodiments in conjunction with the Drawings, of which:

FIGS. 1A and 1B provide portions of images of a conventional portable, transducer-based hand-held contact tonometer device (images available at www.ikisstc.com/products/refurbished-mentor-tono-pen-xl.html?sku=REF-MENTOR&gclid=EAIaIQobChMI2MfGhq2x8gIVMgnnCh0TiwDtEAQYAyABEgKddvD_BwE).

FIGS. 2A, 2B schematically illustrates a portion of the tip of a probe of a device of FIGS. 1A, 1B (or an operationally similar device) with a substantially planar front cornea-contacting surface.

FIG. 3 provides an image of a portion of a conventionally structured ultrasound pachymeter of related art.

FIGS. 4A, 4B present OCT images of the cornea providing evidence of wrinkling of the inner fibers and endothelium of the cornea during the applanation procedure as well as the increased corneal thickness due to applanation.

FIGS. 5A, 5B, 6A, and 6B schematically illustrate related but not mutually exclusive embodiments of the present invention.

FIGS. 7A, 7B provide schematic cross-sectional illustrations of related embodiments of the tips of the transducer probe for use with a contact ultrasound pachymeter, structured according to the idea of the invention.

FIG. 8 illustrates schematically a retrofit-based transformation of the conventional tip of the conventional transducer probe with a related embodiment of the invention to configure a hybrid/composite transducer probe according to the idea of the invention.

FIGS. 9A, 9B provide schematic illustrations of an embodiment of the invention, in cross-sectional views.

FIG. 10 provides an image of a hybrid transducer probe of a contact tonometer.

FIG. 11 provides an image of a hybrid transducer probe of an ultrasound pachymeter.

FIG. 12 is a cross-sectional view of a probe tip cover structured according to an embodiment of the invention.

FIG. 13 is another view of the embodiment of FIG. 12.

FIG. 14 is a perspective view of a portable contact transducer-based tonometer apparatus with the probe tip covered with an embodiment of a cover (compared with FIGS. 1A, 1B)

Generally, the sizes and relative scales of elements in Drawings may be set to be different from actual ones to appropriately facilitate simplicity, clarity, and understanding of the Drawings. For the same reason, not all elements present in one Drawing may necessarily be shown in another.

### SUMMARY OF THE INVENTION

Embodiments of the invention provide a transducer probe judiciously configured for use with a contact ophthalmological instrument equipped with and relying in operation on a transducer. The transducer probe contains a probe body having a tip. The probe body has a body axis; a first portion of the probe body including a front transducer surface; a second portion of the probe body circumscribing and forming a ridge above the first portion of the probe body. The probe body further has a front probe body surface that is transverse to the body axis and dimensioned to contact the cornea of an eye, the front probe body surface being inwardly shaped. In at least one case, the front transducer surface defines at least an axial portion of the front probe body surface and/or the front transducer surface is substantially opaque to light. In at least one implementation, the front transducer surface may be circumscribed with a ring-shaped layer of material different from a material of the front transducer surface; and/or the front transducer surface may be separated from the second portion of the probe body, in a radial direction, with such ring-shaped layer. Alternatively or in addition, substantially every implementation of the transducer probe may be configured to have a cornea-contacting surface of the second portion of the probe body that has a first radius in a plane transverse to the body axis and a first non-zero surface curvature with a first sign, and/or that is dimensioned to be substantially tangentially-parallel with the front transducer surface along a perimeter of the front transducer surface.

Practically every implementation of the transducer probe can optionally be configured such that the second portion of the probe body is reversibly separable and removable from the first portion of the probe body. In this case, for example, the second portion of the probe body may include an article of manufacture having an article body with an article axis and a front surface of the article body; and a hollow in the article body that extends throughout the article body along the article axis and defines an aperture in the front surface of the article body. (Optionally, the article body in this case may have a substantially cylindrical outer surface and/or a substantially conical outer surface. Optionally, in this case, the hollow of the article body may be substantially cylindrically shaped; and/or a surface of the hollow and a surface of an outer surface of the article may be substantially co-axial with one another; and/or a front surface of the article body may be rotationally-symmetric about the article axis.) The hollow of the article body may be dimensioned to accommodate at least the first portion of the probe body therein.

Alternatively or in addition, and substantially in every implementation, the front transducer surface may incorporate or be connected with a piezo or pressure sensor element; and/or have a second radius in a plane transverse to the body axis and a second non-zero surface curvature with the first sign. Alternatively or in addition, a surface of the second portion of the probe body and the front transducer surface may be dimensioned to be tangentially parallel to one another substantially at every point of a perimeter of the front transducer surface.

Preferably, the first sign is equal to a sign of a curvature of a surface of the cornea. and/or each of the front probe body surface and the front transducer surface is axially-symmetric about the probe body axis (optionally, the front transducer surface defines a portion of a spherical surface).

In at least one specific embodiment, a front surface of the ridge surrounding the first portion of the transducer probe has a third surface curvature with a third sign, the third sign being opposite to the first sign, the front surface of the ridge being substantially co-axial with the front transducer surface and/or the front transducer surface is made substantially opaque to light when the probe configured as a tonometer probe for use with a hand-held portable contact tonometer.

In at least one case, when the probe configured as a probe for use with a hand-help portable ultrasound pachymeter, the front transducer surface is made substantially transparent to ultrasound.

Embodiments of the invention additionally provide a method for measuring an intraocular pressure (IOP) of an eye, which method includes using a contact tonometer with a transducer probe structured according to any of the above-identified embodiments (or equipping a contact tonometer with such transducer probe and then using the so-equipped contact tonometer); mechanically connecting at least the front surface of the transducer probe (which represents the front transducer surface) with the cornea; and operating the transducer to take a measurement of the IOP (without transmitting light through the front transducer surface and/or a first portion of a probe body and/or a second portion of the probe body towards the cornea). The step of equipping may include includes removably positioning the first portion of the probe body into an axially-extending hollow of a second portion of the probe body such that a) a portion of a front surface of the first portion of the probe body is substantially in contact with a reference element of the hollow and/or b) the second portion of the probe body is located to circumscribe the first portion of the probe body and to form a ridge above at least the front transducer surface. Alternatively or in addition - and substantially in every implementation of the method - the step of equipping may include placing a front surface of the second portion of the probe body to be substantially tangentially-parallel with the front transducer surface along a perimeter of the front transducer surface and/or the method may optionally include a step of forming a barrier to transfer of microorganisms between the eye and the probe body.

The latter step, when present, may be carried out by removably positioning an elastic cover over the transducer probe to spatially-coordinate a first central area of the elastic cover with a front surface of the probe body surface. (Here, the elastic cover may be structured as a flexible thin film tubular body having an open end and a closed end, a tip portion defining the closed end of the tubular body, and a wall portion connecting the closed end with the open end. The tip portion of the tubular body includes a first central area that has an inner surface and an outer surface, the outer surface being concave.) In one specific case, the step of removably positioning may optionally additionally include securing the elastic cover on the probe body by placing a retention ring of the elastic cover into a groove of the probe body.) In at least one case, the inner surface of the first central area of the tip portion of the elastic cover may outwardly shaped as seen from the open end of the elastic cover, in which case the step of removably repositioning includes placing the front probe body surface (that is inwardly shaped) substantially in contact with the outwardly curved inner surface of the first central area. Generally, the inner surface of the first central area is dimensioned to be substantially convex and the front probe body surface is substantially concave, in which case the step of removably repositioning includes substantially congruently superimposing such inner surface with the front probe body surface.

The step of operating of the contact tonometer includes repositioning the front transducer surface with respect to the cornea and/or repositioning both the first portion of the probe body and the second portion of the probe body with respect to the cornea and/or pressing the front transducer surface and the second portion of the probe body into the cornea. Substantially in every implementation, the method is configured to have a step of mechanically connecting at least the front surface of the transducer probe with the cornea include (i) establishing a direct physical contact between the front transducer surface and the cornea; or (ii) mechanically connecting the at least transducer surface with the cornea through a thin-film of elastic material disposed therebetween. Substantially in every embodiment of the method, the step of equipping the contact tonometer with the embodiment of the transducer prober may include equipping the contact tonometer with the probe having such a front surface that is substantially opaque to light.

Embodiments also provide a contact tonometer system, which system includes a transducer probe that structured according to any of the above-identified embodiments and - in at least one specific case - optionally contains a disposable article of manufacture dimensioned to cover the transducer probe. (Such disposable article, when present, may include a flexible thin film tubular body having an open and a closed end, a tip portion defining the closed end of the body, and a wall portion connecting the closed end with the open end. Here, the tip portion includes a first central area that has an inner surface and an outer surface, the outer surface being concave. Such article is configured to create a barrier, when installed onto the probe tip, to transfer of microorganism between an eye of a patient and the tip of a probe of the contact instrument during a contact ophthalmological examination while at the same time not impeding a measurement of a target parameter of the eye through such article.) Alternatively or in addition - an in at least one embodiment of the contact tonometer system - (a) the inner surface is either convex or substantially planar as viewed internally to tubular body from the open end; or (b) the inner surface is concave as viewed internally to the tubular body from the open end (and, in either of these two cases, the outer surface of the first central area of the tip portion is dimensioned to substantially conform to the corneal surface of an eye). Generally - and substantially in every implementation when the disposable article of manufacture is used, such disposable article may be configured to have an ultimate elongation from about 500% to about 1000% and/or a tensile strength from about 1000 psi to about 5500 psi and/or a modulus of elasticity at 100% strain from about 50 psi to about 2000 psi; and/or be formed from a material including one or more of the following: polyurethane, polyethylene, polypropylene, polyisoprene, polychloroprene, nitrile, and silicone.

Embodiments of the invention additionally provide a method for measuring a thickness of the cornea of an eye, the method including a step of either equipping an ultrasound pachymeter with a transducer probe structured according to those of the embodiments discussed above that satisfy the requirements of ultrasonic pachymetric or using the ultrasonic pachymeter already equipped with the appropriate transducer probe. The method further requires establishing a mechanically connection between a front probe body surface with the cornea, and transmitting an ultrasound wave through the front probe body surface into the eye. Finally, the method includes the step - performed with the use of a programmable processor - of determining a value of the thickness of the cornea based on ultrasound pulses reflected back to and received by the transducer from a surface of the eye and through the front probe body surface. The step of equipping may include removably positioning the first portion of a probe body of the transducer probe into a hollow axially extending throughout a second portion of the probe body of such probe and/or placing a front surface of the second portion of the probe body to be substantially tangentially-parallel with the front transducer surface along a perimeter of the front transducer surface (present at the front surface of the first portion of the probe). Alternatively or in addition - and substantially in every implementation - the method may include a step of removably positioning an elastic cover over the transducer probe to spatially-coordinate a first central area of the elastic cover with a front probe body surface. Here, the elastic cover is configured as a flexible thin film tubular body having an open end and a closed end, a tip portion defining the closed end of the tubular body, and a wall portion connecting the closed end with the open end (the tip portion of the tubular body includes a first central area that has an inner surface and an outer surface, the outer surface being concave). When such elastic cover is used, the step of removably positioning such cover may include securing the elastic cover on the probe body by placing a retention ring of the elastic cover into a groove of the probe body. The inner surface of the first central area of the tip portion of the elastic cover may be outwardly shaped (as seen from the open end of the elastic cover), in which case the step of removably repositioning may include placing the front probe body surface that is inwardly shaped substantially in contact with the outwardly curved inner surface of the first central area. Alternatively or in addition, and substantially in every implementation of the method the mechanically connecting may include either establishing a direct physical contact between the front transducer surface and the cornea or mechanically connecting at least the transducer surface with the cornea through a thin-film of elastic material disposed therebetween.

Embodiments also provide an ultrasound pachymeter comprising a transducer probe configured according to each of the above-discussed embodiments that lend themselves for use with the ultrasound pachymeter. In at least one specific case, such ultrasound pachymeter may additionally be equipped with a disposable article of manufacture dimensioned to cover such transducer probe (the disposable article includes a flexible thin film tubular body having an open and a closed end, a tip portion defining the closed end of the tubular body, and a wall portion connecting the closed end with the open end; and the tip portion includes a first central area that has an inner surface and an outer surface, the outer surface being concave. The disposable article is configured to create a barrier (when installed onto the probe tip) to transfer of microorganism between an eye of a patient and the tip of a probe of the contact instrument during a contact ophthalmological examination while at the same time not impeding a measurement of a target parameter of the eye through such disposable article. In at least one implementation of the ultrasound pachymeter employing the disposable article, the inner surface of the first central area of the tip portion of the article may be convex or substantially planar as viewed internally to tubular body from the open end, while the outer surface of the same first central area is dimensioned to substantially conform to the corneal surface of an eye. Substantially in every implementation of the ultrasound pachymeter employing such disposable article, the disposable article may be configured to have an ultimate elongation from about 500% to about 1000% and/or a tensile strength from about 1000 psi to about 5500 psi and/or a modulus of elasticity at 100% strain from about 50 psi to about 2000 psi and/or be formed from a material including one or more of the following: polyurethane, polyethylene, polypropylene, polyisoprene, polychloroprene, nitrile, and silicone.

A skilled artisan will readily appreciate and understand further details of the embodiments of the invention from the discussion(s) below.

### DETAILED DESCRIPTION

As was already alluded to above, the measurements of the IOP and the corneal thickness provide two very important data readings that serve to derive conclusions about the status of health of the eye.

### Intraocular Pressure and Conventional Hand-Held Contact Tonometer

Regardless of whether a hand-held contact tonometer (of a TonoPen® or Mackay Marg variety, for example) or a Goldmann-type tonometer is used for measurement of the intraocular pressure, the measurement is performed according to the principle of applanation tonometry, in which the IOP is inferred from the force required to flatten (applanate) a pre-defined area of the cornea with the tip of the probe of the tonometric instrument. It is the contact pressure required for flattening of such area of the cornea that is used as a measure of the IOP.

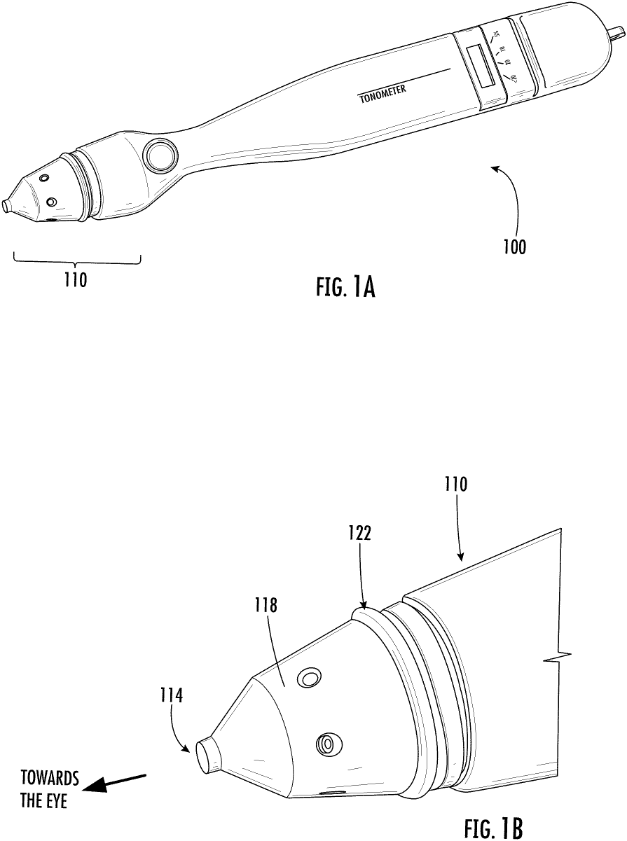

The use of an electronic Tono-Pen®-type portable tonometer, for example (an image of which is presented in FIG. 1A) facilitates a quick measurement of IOP in the eye clinic without the use of light (as compared with the use of a Goldmann-type tonometer, which requires the use of light for imaging of the cornea as an instrument for the measurement of the IOP which light is transferred through the optically-transparent probe of the tonometer to the cornea), and can be performed by the technician. Practice shows, however, that the use of this tonometer almost always produces the values of IOP that are substantially lower than the those identified with the use of a more accurate Goldmann-type tonometer-based IOP measurement, which measurement admittedly requires much more expertise and clinical time to measure. (For description of methodologies utilizing a Goldmann-type tonometer, the reader is referred to, for example US D775,736; US 10,463,251; US 11,026,576; WO 2016/167827, the disclosure of each of which is incorporated herein by reference).

FIG. 1A provides an image of one of widely used portable, hand-held contact tonometers (often referred to as a TonoPen® or a similar one). These devices are structured to perform tonometric measurements (leading to determination of intraocular pressure, IOL) in a well-recognized fashion by applanating the cornea at the time when the front, cornea-contacting surface of the tip of the optically-opaque probe of the device is brought in contact with the cornea, and determine the applanation pressure from the reading of the pressure transducer a portion of which is built into the tip of the probe. The tip 110 of the probe has a planar front, cornea-contacting surface. The front portion of the device of FIG. 1A is shown in FIG. 1B, where the tip 114 of the probe 110 of the device 100, as well as at least a portion of the body of the probe 110 itself, are shown to be optionally covered with a thin-film cover 122 (the lead-line 122 is drawn to point to the retaining ring portion of the cover) such as the one discussed, for example, in US 7,287,856, the disclosure of which is incorporated herein by reference). The front surface 114A of the tip 114, configured to contact the corea in operation, is conventional planar.

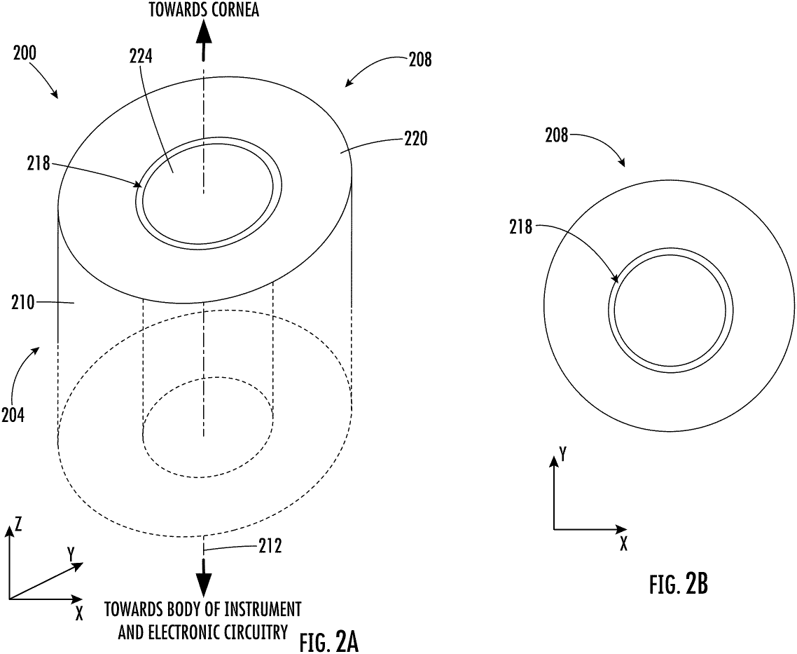

While various incarnations of the contact hand-held tonometers exist, the tips of the probes such devices are typically structured according to the same principle that is illustrated schematically in FIGS. 2A, 2B.

FIG. 2A shows a sketch of a portion of a tip 200 of a transducer probe of a conventional portable tonometer of related art (such as a TonoPen 100), which corresponds to the tip 114 of FIG. 1A. The tip 200 includes a body 204 (the precise outer shape of which is substantially irrelevant for the purposes of this discussion) terminated with a front surface 208, which is configured to be brought to touch and press into the cornea of an eye subject to the tonometric examination - either in direct contact (such that the substantially planar front surface 208 comes in direct physical contact with the cornea) or in indirect contact when the surface of the cornea and the surface 208 are pressed against one another but are physically separated by a film of the cover 122. The body 204 typically includes an outer body portion, marked as 210. The substantially planar front surface 208 contains the axial substantially planar portion 224 and the surrounding / circumscribing it substantially planar portion 220. The surface portion 224 is at least a part (at least a surface) of the transducer of the contact tonometer 100, and the surfaces portions 220, 224 are conventionally separated, in the plane of the front surface, by a functional gap 218.

This small functional gap 218 may be generally filled with a pre-defined material. However, and regardless of the specifics of the material structure of the functional gap 218, the space between the portions 220 and 224 of the tip 200 is configured to facilitate the operation of the device. For the purposes of this disclosure, the term transducer is defined to denote a device that receives a signal in the form of one type of energy and converts it to a signal in another form. Such transducer, depending on the specifics of a particular implementation, may include a plunger or piston of sorts (which may be made repositionable within the body 204) and/or the transducer may be configured as a pressure transducer and include a piezo element separated from the outer body portion 210 at least with the ring of the predefined material perceived from outside of the probe as the ring-shaped region 218.

As shown in FIG. 2A, both the front surface portion 220 of the outer body portion 210 and the element/part 224 of the transducer are located substantially at the same level, maintain the planar nature of the overall front surface 208. FIG. 2B shows the front surface of the tip 200 in a top view.

The surface 208 (including regions 218, 220, 224) is substantially optically opaque, and no light is used in operation of the tonometer 100 (or a similar tonometer). Instead, in operation, the tip 200 of the probe of the transducer of the tonometer is brought in contact with the cornea at the surfaces 208, 220, 224, and the pressure applied to the cornea by the surface 224 of the transducer and by the transducer-surrounding surface 220 causes applanation of the cornea. The transducer data is further related to the instrument through the available electronic paths (optionally built-in the body of the embodiment 200; not shown) and, with the use of the appropriately programmed electronic circuitry such as a programmable processor, the calculations of the IOP are then performed as required.

The tip 200 or a tip of the transducer probe of a contact tonometer structured substantially similarly to the tip 200 will be referred herein as an original tip or a conventional tip. Similarly, the transducer probe having such a tip may be referred to as an original probe or a conventional probe.

### Corneal Thickness and Conventional Ultrasound Pachvmeter

As far as the measurement of the corneal thickness is concerned, a typical conventional ultrasound pachymeter (such as the one fabricated by the DGH technology, Inc., for example; see dghtechnology.com/Auriga/wp-content/uploads/2020/08/DGH-55B-INS-OMENG-R3-DGH-55B-Operator-Manual-English.pdf, which publicly available document is incorporated herein by reference) shown in FIG. 3 includes a hand-held handle 300 with at least a portion of its transducer housed in a transducer housing portion 320 and a probe of the transducer (or transducer probe) 310, a housing of the instrument, and various accessories and convenience features. The very tip 310A of the transducer probe 310 has a planar front, cornea-contacting surface. The handle may include a piezoelectric crystal configured to emit an ultrasound wave at about 20 MHz through the planar surface of the tip, which in operation is touched to (brought in contact with, pressed against) the cornea.

A tip of the transducer probe of a contact ultrasound pachymeter structured substantially similarly to the probe 310 may be referred herein as an original probe or a conventional probe. Similarly, the tip of such conventional probe may be referred to as an original tip or a conventional tip.

The general principle of operation of such pachymeter is as follows: The planar surface of the (often polystyrene) tip of the ultrasonic transducer probe is placed in contact with the patient’s cornea, which automatically initiates a measurement cycle. At the start of the measurement cycle, the electronic circuit board transmits voltage pulses to the ultrasonic transducer (probe). The piezoelectric element in the transducer converts these voltage pulses into ultrasonic energy, sending a pulse of a high frequency sound waves (20 MHz damped to 13 MHz, for example) through the eye, and reflected pulses (echoes) are received back to the transducer and are converted to voltage pulses. The first echo to be received comes from the anterior corneal surface. If an echo spike from the anterior corneal surface is received within an anticipated time window, the device then prepares to receive an echo spike from the posterior corneal surface. Only anterior and posterior echo spikes that fall within specified voltage limits that ensure that the probe tip is perpendicular to the cornea surface are accepted for processing. The time interval between the accepted anterior and posterior echo spikes represents the thickness of the cornea. The time interval is converted to a corresponding distance, or thickness, based on the acoustic velocity through the cornea.

There are two methods by which pachymeter creates an average reading. (1) A pulse locked method: here, the device will record all readings that are made with the pachymeter tip being within about 5 degrees to 10 degrees of deviation with respect to the normal to the cornea, rejecting those outside the range; and (2) a fixed number of consecutive readings is taken with the above-specified angular orientation of the tip before the results are averaged. If the probe is not perpendicular or the readings are too disparate, the series of measurements data point is rejected, and the measurement must be re-taken.

As explicitly discussed by the manufacturer of the device, proper applanation of the cornea is necessary for obtaining an accurate measurement. Proper applanation occurs when the flat tip of the probe comes into full contact with the cornea perpendicular to the cornea surface. The user generally should ensure that pressure against the cornea is minimized, however.

Ultrasound pachymetry is known as a methodology for a quick measurement of the thickness of the cornea in the eye clinic, completed by the technician. The errors produced by ultrasound pachymetry measurement are almost always significantly greater than those of the more accurate OCT-based pachymetry measurement (the latter approach requiring much more expense and clinic time to be completed).

### Deformation of the Cornea in Operation of Pachvmeter / Tonometer

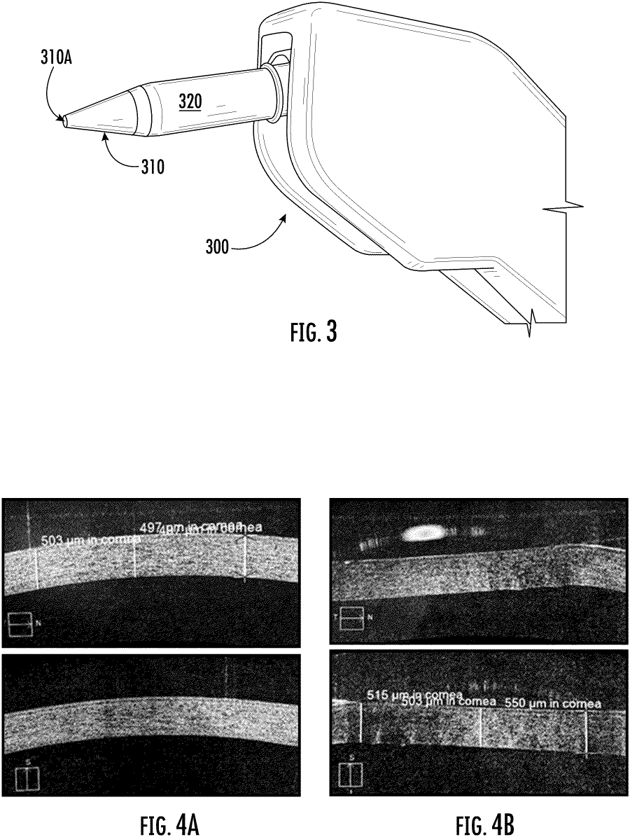

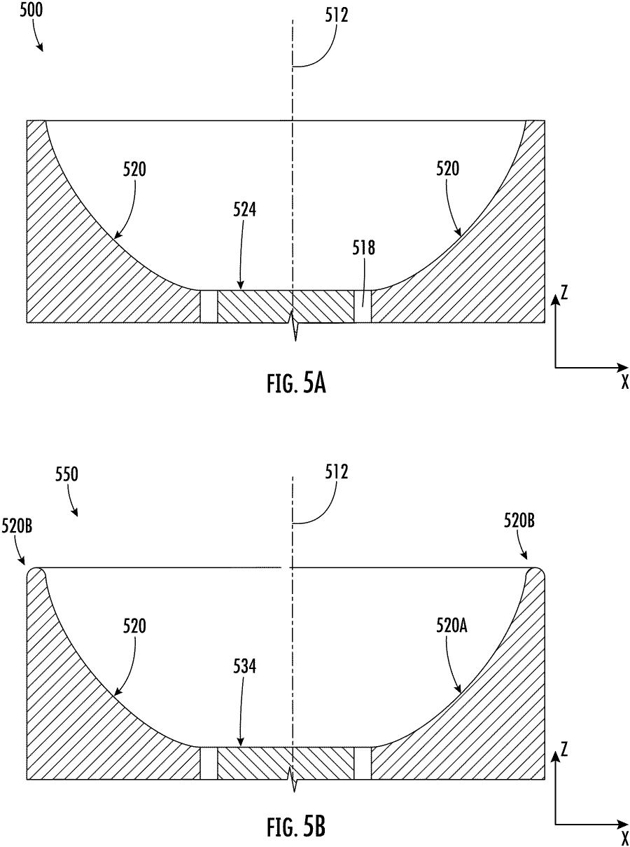

As a skilled person will readily recognize, during the process of applanation of the cornea with the use of substantially any device - whether the ultrasound pachymeter 300 of FIG. 3 or the contact hand-held tonometer 100 with an optically-opaque tip of the probe 110/200, the thickness of the cornea increases at least in some areas. The inner corneal fibers actually buckle or wrinkle as a skilled artisan will undoubtedly deduce from the OCT images of the cornea of FIGS. 4A, 4B (taken of the same cornea - in one case applanated, FIG. 4B, and in another non-applanated, FIG. 4A). Based on the analysis of these OCT images of FIGS. 4A, 4B, a skilled person will appreciate the comparative wrinkling of the inner fibers and endothelium with applanation as well as the increased corneal thickness due to applanation. The wrinkled corneal fibers indicate the “buckling” of the cornea away from the central, axial portion of the cornea-contacting surface of the tip of the probe (where the pressure gradient across the applanated surface is at a minimum in the very center of the applanated circle where the sensor portion of the tonometer’s tip is measuring the pressure or where the ultrasound transferred from the transducer through the pachymeter’s probe reaches the cornea). This, even by itself, is a cause of the error of the very measurement being performed.

This buckling of the cornea tends to pull the cornea away from the applanating surface in the center, thereby not only resulting in a substantially non-uniform pressure applied by the surface of the tip of the tonometer’s probe to the cornea (and vice versa, when the tonometric measurement is performed) - lower in the center, higher in a peripheral portion of the cornea-contacting surface of the tip of the probe - but also causing the actually measured values of intraocular pressure to be understandably reduced . Even notwithstanding this practical evidence, the very process of the IOP measurement with the use of a Tono-Pen® type portable tonometer 100, for example, remains operator dependent: the harder a less-trained operator pushes on the cornea, the more the device underestimates the value of the IOP as compared to the measurement performed with the use of a Goldmann-type tonometer. Similarly, this unpredictable deformation of the cornea during the use of a conventional ultrasound pachymeter 300 not only introduces the error in the measurement of the corneal thickness but necessarily makes the results of the measurement operator-dependent: the harder a less-trained operator pushes on the cornea, the more “buckling” of the cornea and, therefore, error of the measurement, occurs.

Embodiments structured according to the idea of the invention address the above-identified problems persistently present when either the contact hand-held tonometer is used or when an ultrasound pachymeter is used.

Accordingly, the problem of performing the measurement of intraocular pressure with the use of a contact Tono-Pen® type tonometer in absence of light reflected from the eye through the probe and/or tip of the probe (for example, with the use of a pressure transducer) and without the need to correct for the contribution of (at least) corneal thickness and stiffness is solved by devising a transducer probe the cornea-contacting front portion of which includes surface(s) that generally define an inwardly-caved or inwardly-shaped shape (that is such a shape of the cornea-contacting front portion that defines a cave in a body of the transducer probe, is scooped in, or caved in in the central axial portion of the probe as compared to a radially-extended portion of the probe). In other words, the tip of the probe is shaped to have a radially-extended cornea-contacting surface of the tip be raised above a central, axial portion of the cornea-contacting surface of the tip that corresponds to the location of the probe sensor/transducer. (In one specific case, the tip is appropriately shaped to have a first portion having a concave shape. In another related case, a portion of the tip configured to surround the central, axial portion of the front surface of the tip of the transducer probe as a ridge elevated over such central portion.) In a similar fashion is solved the problem of errors of the measurement of the corneal thickness with the use of a contact ultrasound pachymeter, by utilizing a pachymeter probe the cornea-contacting front portion of which includes surface(s) that generally define an inwardly-caved shape.

Notably, as the skilled person will readily understand from the following discussion, the target configuration of the transducer tip of an instrument at hand is achieved in a variety of fashions, including, in one case, a direct modification of the shape of the original tip of the probe itself. In another case, a hybrid / composite / compound tip of the transducer probe is formed by complementing the already existing conventional flat front surface tip of the probe with a judiciously shaped add-on contraption to produce a hybrid / composite / compound transducer probe with a hybrid / composite / compound tip the front surface of which is desirably inwardly shaped.

### EXAMPLES OF EMBODIMENTS OF THE INVENTION

As described in the following disclosure in more detail on the specific examples, an embodiment of an invention (pertaining to a contact ophthalmological instrument such as either the ultrasound pachymeter or a contact hand-held tonometer, each of which necessarily employs a transducer) provides a probe of the transducer of the instrument that has a tip and that is configured for use with such contact ophthalmological instrument. The probe of or for the transducer of such instrument includes a probe body that has a body axis. In stark contradistinction with a probe configured for use with a Goldmann-type tonometer (which by the very nature of it is devoid of a transducer, as the principle of operation of the Goldmann tomoneter does not utilize transformation of an input signal in the form of optical energy into a signal in another form of energy), an embodiment of the discussed probe includes a transducer (that is, a device in operation transforming a signal in one form of energy to a signal in another). The transducer probe also includes a first portion of the probe body and a second portion of the probe body circumscribing and forming a ridge above the first portion of the probe body. A front probe body surface is inwardly shaped and transverse to the body axis and is dimensioned to contact the cornea of an eye. Substantially in every implementation, at least an axial portion of the front probe body surface may be structured to define the front surface of the transducer (interchangeably referred to as a front transducer surface), and in at least one specific case the front transducer surface may be configured to be substantially opaque to light.

It is understood that, going forward, a discussed embodiment of the probe of the transducer of a contact ophthalmological instrument is considered to represent a probe of either a contact tonometer or an ultrasound pachymeter unless expressly stated otherwise.

### Example 1. The Inwardly-Shaped Surface of the Tip of the Transducer Probe of a Contact Tonometer.

Considering now only a specific case of when a second portion of the probe body (circumscribing the central, axial area of the front surface of the embodiment of the probe) has an inwardly shaped or even specifically concave front surface, such front surface of the second portion of the probe body may be shaped to be tangentially-parallel with the front surface of the transducer at least along the perimeter of the surface of the transducer. In at least one case, the front surface of the transducer may be also curved such that the signs of curvatures of the front surface of the transducer and the front surface of the second portion of the probe body are the same. In a rather specific case, the second portion of the probe body may additionally include a peripheral surface portion encircling the curved front surface of the second portion and having a curvature with a sign opposite to the sign of the curvature of the first portion. In this specific case, the curved front surface of the second portion of the probe body and the curved peripheral surface portion are configured to merging tangentially with one another along a closed plane curve.

To this end, FIGS. 5A, 5B illustrate one embodiment of the transducer probe of a contact tonometer that can be thought of a judiciously modified - according to the idea of the invention - original tip of the transducer probe of the related art to make it inwardly-shaped in a fashion similar to or at least remotely resembling the change of spatial elevation or profile of the typical cornea.

FIGS. 5A, 5B illustrate related embodiments of the transducer probe 500, 550 of the invention. Similarly to the structure of the conventional tip 200 of FIG. 2A, the axial, front transducer surfaces 524 of the embodiments remain substantially planar, while the front surfaces 520 of the outer portions of the probe body is inwardly curved and dimensioned to be merging with the surface 524 in a substantially tangentially-parallel fashion along the corresponding perimeters and/or corresponding edges of these surfaces. The outer portion of the probe body circumscribes and forms a ridge over / above the first portion of the probe body containing the surface 524, in each of the embodiments. The embodiments 500, 550 differ in one detail: the front surface of the outer portion of the probe body is shown sub-divided into the inwardly-curved surface 520A (which is tangentially-parallel to the axially-located front surface of the transducer 524, as discussed above) and the peripheral surface 520B, which is tangentially merging with the surface 520A along a closed curve. The sub-surfaces 520A, 520B have corresponding curvatures the signs of which are opposite to one another. Preferably - and in at least one implementation - each of the surfaces 520, 520A, 520B, 524 is rotationally symmetric about the axis 512 of the embodiment. The numeral 518 denotes a ring-shaped layer of material different from the material of the transducer surface 524. Understandably, the axial portions of the embodiments 500, 550 of the transducer probe of the invention corresponding to and limited by the ring-shaped material 518 represent first portions of the probe body, while the portions lying outside of the ring-shaped material 518 (and, therefore, containing respectively surface 520 and 520A) represent second portions of the probe body.

In operation, at least the surface 524 is brought in contact with the cornea of an eye subject to the tonometric measurement (which means and is defined as juxtaposing the surface with and/or against the cornea directly, by touching immediately, or indirectly, with a film of the cover such as the cover 122 between the tip 500, 550 and the cornea, when such cover is present). After, the portable tonometer of which the tip 500, 550 is a part is operated in a conventional fashion to measure the IOL with the use of the contact transducer-containing tonometer.

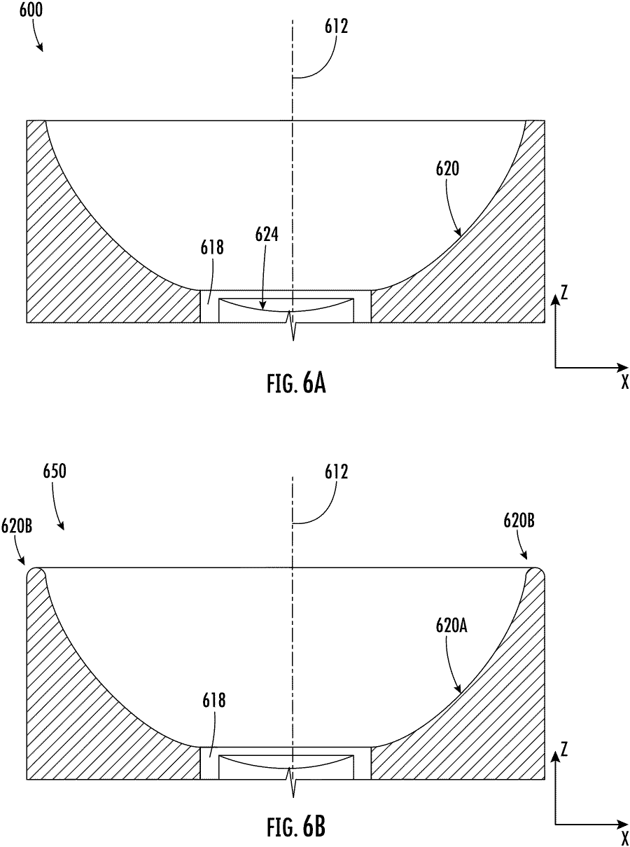

FIGS. 6A, 6B illustrate a related embodiment of the transducer probe of the invention, in which the axially located front transducer surface is also judiciously spatially curved. These Figures schematically depict, in cross-sectional views, related embodiments 600 and 650 of the tip of the transducer probe structured according to the idea of the invention. Here, the front surface of the overall tip 600 is composed of the still axially located but now spatially-curved front transducer surface 624 of the first portion of the probe body and the front surface 620 of the outer body portion that circumscribes the surface 624 and forms the ridge over it. In stark contradistinction with the related art of FIGS. 2A, 2B, both of the surfaces 620 and 624 are now curved inwardly (towards the instrument to which the probe is attached) in a fashion similar to that of a typical cornea. At the same time, the surfaces 620, 624 are dimensioned to be substantially tangentially-parallel to one another along their respectively-corresponding perimeters (the inner perimeter of the surface 620 and the outer perimeter of the surface 624) and/or corresponding edges of these surfaces. The numeral 618 denotes a ring-shaped layer of material different from the material of the transducer surface 624. Understandably, the axial portions of the embodiments 600, 650 of the transducer probe of the invention corresponding to and limited by the ring-shaped material 618 represent first portions of the probe body, while the portions lying outside of the ring-shaped material 618 (and, therefore, containing respectively surface 620 and 620A) represent second portions of the probe body.

In operation, at least the surface 624 is brought in contact with the cornea of an eye subject to the tonometric measurement (whether directly, by touching immediately, or indirectly, with a film of the cover such as the cover 122 between the tip 600, 650 and the cornea, when present). After, the portable tonometer of which the tip 600, 650 is a part is operated in a conventional fashion to measure the IOL with the use of the contact transducer-containing tonometer.

The embodiment 650 of FIG. 6B is substantially identical to the embodiment 600 with the exception that the surface 620 is comprised of two sub-surfaces: the sub-surface 620A and a peripheral sub-surface 620B that circumscribes the sub-surface 620A while tangentially merging with it. The curvatures of the sub-surfaces 620A and 620B have opposite signs. Preferably, each of the surfaces 620, 620A, 620B, 624 is rotationally symmetric about the axis 612 of the embodiment.

### Example 2. The Inwardly-Shaped Surface of the Tip of the Transducer Probe of an Ultrasound Pachymeter.

By analogy with shaping of the front surface of the transducer probe of a contact tonometer discussed above in reference to FIGS. 5A, 5B, 6A, 6B, in embodiments of the transducer probe of the ultrasound pachymeter the front surface is similarly configured to be inwardly-shaped (preferably - concave), an in a specific case to have such inwardly-shaped front surface substantially geometrically match the convexly-shaped cornea. In a specific case, the peripheral portion of the front cornea-contacting surface of the embodiment of the invention may be additionally spatially curved with a curvature sign being opposite to the curvature sign of the axial portion of the front surface of the transducer probe.

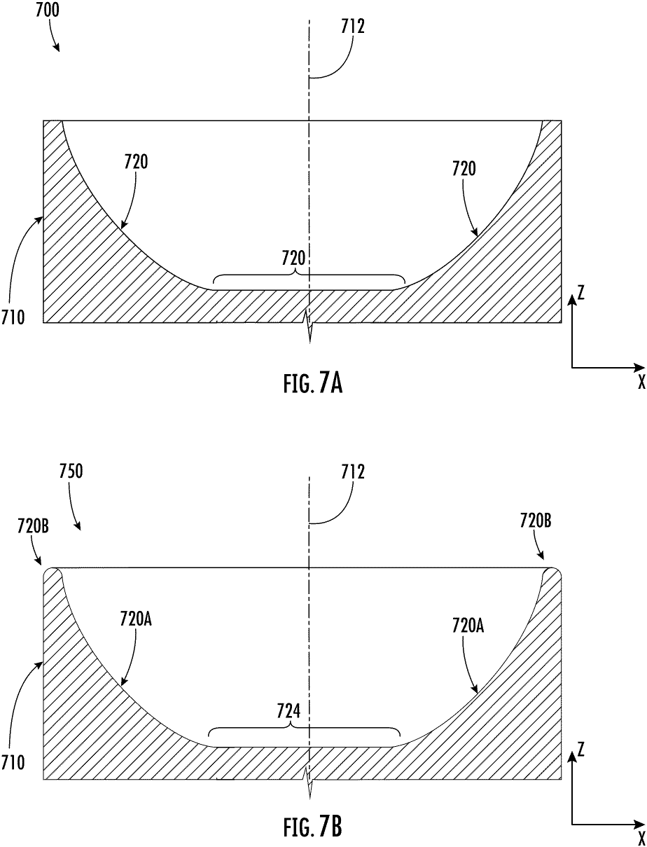

The schematic representations of the related solutions for the transducer probe of the pachymeter are shown in FIGS. 7A, 7B. Here, the front surface of the tip of the probe 700 includes a substantially planar axial, centrally located portion 724 of the transducer surface and an inwardly-curved (preferably - substantially concave) surface portion 720 circumscribing the surface portion 724 and merging with it in a tangentially-parallel fashion. The sign of the curvature of the portion 720 is the same as the sign of the curvature of the cornea. In operation, both the surface portions 720, 724 are brought in contact with the cornea of an eye subject to a pachymetric measurement (whether directly, by touching immediately, or indirectly through a protective film or cover between the embodiment 700 and the cornea). After, the portable ultrasound pachymeter is operated in a conventional fashion to measure the cornea central thickness with the use of the transducer of the instrument.

The embodiment 750 of FIG. 7B is substantially identical to the embodiment 700 with the exception that the curved surface 720 is comprised of two sub-surfaces: the sub-surface 720A and a peripheral sub-surface 720B that circumscribes the sub-surface 720A while tangentially merging with it. The curvatures of the sub-surfaces 720A and 720B have opposite signs. In yet another relate implementation, the axially-located surface portion 724 of the front transducer surface of the transducer probe may be additionally inwardly curved (by analogy with the surface 624) while maintaining the tangentially-parallel relationship between the surface portions 720,720A and 724. Preferably, each of the surfaces 720, 720A, 720B, 724 is rotationally symmetric about the axis 712. Notably, any of the embodiments 700, 750 can be configured to be disposable (that is, separable from and re-attachable to the transducer housing portion 320.)

Understandably, the axial portions of the embodiments 700, 750 of the transducer probe of the invention corresponding to and containing the surfaces 720, 724 respectively represent first portions of the probe body, while the portions lying outside of the radial limits defined by the surfaces 720, 724 (and, therefore, containing respectively surfaces 720, 720A) represent second portions of the transdycer probe body.

### Example 3. Embodiments of the Hybrid/Composite Transducer Probe for a Contact Ophthalmological Instrument.

FIG. 8 presents generalized schematic of the hybrid/composite transducer probe 800 that, in practice, is formed by retrofitting of the original transducer probe (such as 110, 310) with a specific article of manufacture configured as a cap or crown 804 that is juxtaposed with and over the existing conventional tip of conventional tip of the conventional probe (be that a probe of the ultrasound pachymeter or a contact hand-held tonometer).

In this specific example, the substantially rotationally-symmetric crown or cap 810 is fittingly and removably slid (as depicted by the arrow 814) over the existing tip of the transducer probe such as to have the tip at least partially fit into the hollow 816 of the crown 810 and to have the rim or ridge 804 of the crown be appropriately raised above the level defined by the planar cornea-contacting surface (114A in case of the probe 110, 310A in case of the probe 310). The original probe thus forms a first portion of the body of the composite probe, and the crown 810 forms the second portion of the composite probe body circumscribing the first portion of the composite probe body. Notably, the front ring surface of the ridge 804 of the crown 810, when retrofitted over the tip of the conventional probe, is raised over the flat surface of the original, conventional tip. The surface of the ridge 804 is presented schematically, and can generally deviate from that depicted in FIG. 8. It is appreciated that the inwardly-shaped cornea-contacting surface of the so-assembled composite tip of the probe 800 includes a surface of the ridge 804 (raised above the surface 114A or 310A) as well as the planar surface 114A, 310A itself. During the measurement, the front surface of the so-formed and appropriately dimensioned composite tip of the probe 800 is brought in contact with the corneal surface such as to have both the ridge 804 and the planar surface 114A, 310A touch the cornea to at least reduce the degree of “buckling” of the corneal surface during the applanation (see discussion in reference to FIGS. 4A, 4B) or even to substantially not allowing the cornea to buckle and thereby providing a more accurate measurement of the corneal thickness when compared to the conventional pachymeter measurement (or the conventional contact tonometer measurement (despite the fact that the cornea-contacting surface of the composite tip of the composite probe 800 may not be ideally conforming to the surface of the cornea.

In a preferred implementation, of course, the inwardly-caved surface formed by the inner surface of the ridge 804 and the conventionally-planar surface of the tip of the conventional probe 114A, 310 may be configured to contain a generally concave surface portion. This is achieved either by shaping the inner surface of the ridge 804 to be concave (as seen into the cap/crown 810 and towards the probe), or by modifying the conventionally planar surface 114A, 310A to be concave, or both.

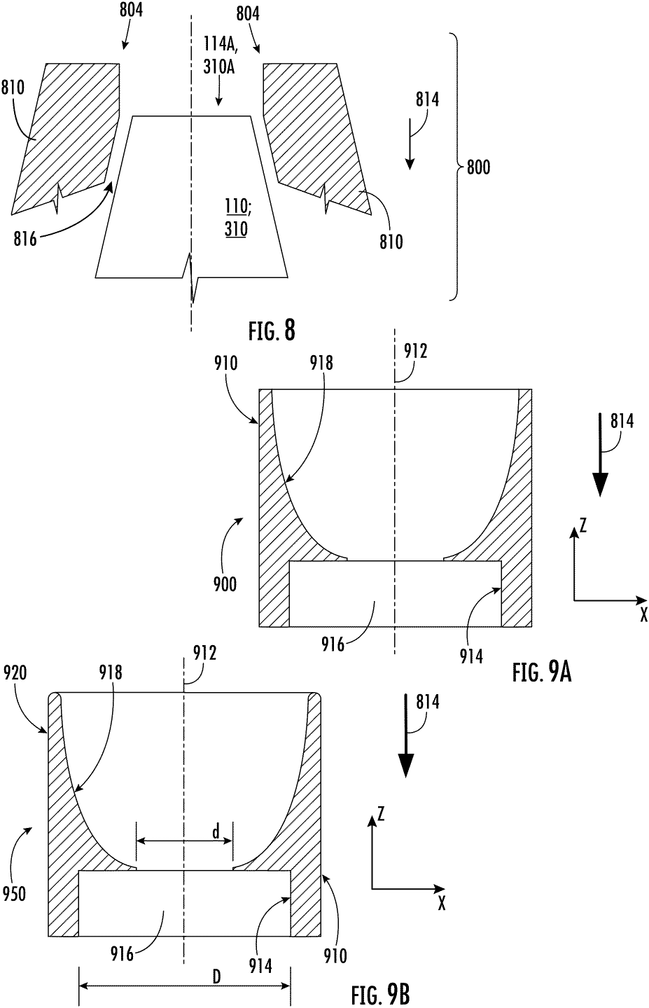

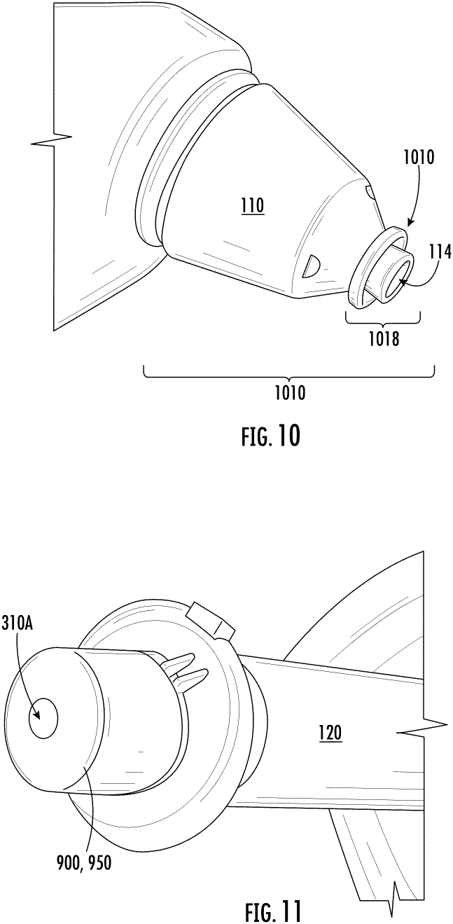

FIGS. 9A, 9B illustrate the first option. Here, FIGS. 9A, 9B present schematics of a “tip cap” or “tip crown” 900, 950 which, when fitted onto the conventionally-structured tip of the probes 110, 200, 310 of the devices of related art (FIGS. 1A, 1B, 2, 3), converts such conventional tip to one shaped according to an idea of an embodiment of the invention. The tip cap or crown 500 is, substantially, an article of manufacture with a preferably cylindrical or conical outer surface 910, and an inner surface 914 judiciously shaped to receive and fittingly match the outer shape of the conventional tip (preferably, a substantially cylindrical or conical). The inner surface 914 may be co-axial with the surface 910 with respect to the axis 912 of the cap 900, 910, and an inwardly-caved (or, in a specific case - inwardly curved or even concave) front surface 918. In practice, the cap 900 is dimensioned such that the conventional tip of a conventional transducer probe 100, 200, 310 precisely fits inside the hollow 916 of the cap 900, 910 and - at least in one specific case - when the conventional tip is appropriately inserted into the hollow 916, the surfaces 224 of the tip 200 and the 918 of the cap 500 may be substantially tangentially-parallel to one another along the corresponding perimeters and/or facing each other edges of these surfaces. FIG. 9B illustrates a related embodiment 950 structured in a substantially similar fashion, except the inwardly-caved surface 918 is circumscribed with the annulus-bound (when viewed in the -z direction) peripheral surface 920 the curvature of which has a sign that is opposite to the sign of the curvature of the surface 918. Understandably, the conventional tip/probe used in combination with the cap/crown of FIGS. 9A, 9B represents the first portion of the body of the resulting hybrid transducer probe while the cap/crown itself represents the second portion of the transducer probe body.

In reference to the specific structure of the conventional contact tonometer tip of FIGS. 2A, 2B, in each of FIGS. 9A, 9B the dimension D represents a value slightly higher (as required for proper mechanical fitting) than the outer diameter of the surface 220, and the dimension d represents the outer diameter of the surface 224.

Accordingly, an embodiment of the invention provides a method for modifying a conventionally planar-surfaced tip of a transducer probe of a contact hand-held tonometer (of a Tono-Pen® variety or that operating according to a similar principle(s)) and/or of the conventional ultrasound pachymeter.

A skilled person having the advantage of this disclosure will appreciate that existing Tono-Pen® type tonometer devices and/or existing ultrasound pachymeters of related art can be retrofitted with an embodiment of the tip structured in a fashion discussed above (FIGS. 9A, 9B), or in a similar fashion as long as the overall, composite probe is shaped to have a radially-extended cornea-contacting surface of the probe be raised above a central, axial portion of the cornea-contacting surface of the probe that corresponds to the location of the probe sensor/transducer.

Notably, any of the embodiments 900, 950 can be configured to be disposable (that is, separable from and re-attachable to the conventional tip of the transducer probe.)

A preliminary prototype of an embodiment of the composite probe 1000, formed by adding the “crown” to and over the conventional flat-front-surface tip of the Avia Tonopen is shown in FIG. 10. Here, the substantially rotationally-symmetric crown 1010 (embodiments of which are schematically illustrated in FIGS. 9A, 9B) is fittingly and removably positioned over the existing tip 114 such as to have the rim or ridge 904 of the crown be appropriately raised above the level defined by the planar cornea-contacting surface 114A of the tip114. (Note the outer ring retrofitted over the tip is raised over the flat surface of the original tip.) In combination with the crown or cap 1010, the conventional flat-front-surface transducer probe 110 formed an embodiment of the composite / hybrid transducer probe 1000 structured according to an embodiment of the invention. In operation, the front surface of the composite tip of this composite probe is brought in contact with the corneal curvature to at least reduce the degree of “buckling” of the corneal surface during the applanation, or even to substantially not allowing the cornea to buckle and thus providing a more accurate IOP when compared to even the conventional Goldmann-tonometer-based measurement despite the fact that the cornea-contacting surface of this composite tip is not ideally conforming to the surface of the cornea.

Proof of workability of an embodiment of the invention of FIGS. 9A, 9B, 10 (denoted as “CATS” in the table below) was provided by measuring IOP in 46 patients’ eyes with a Tono-Pen® type device by various technicians and then by a physician. Following these measurements in a given eye, the IOP was measured by the physician with the Tono-Pen® type tonometer a tip of which as modified according to the idea of the invention. For comparison of the accuracy, the IOP was also measured with the Goldmann tonometer using both a standard GAT tip and a CATS tip (see, for example, US 10,463,251 or US 11,026,576, the disclosure of each of which is incorporated herein by reference). The table below illustrates the variability of results between the technician and physician operators (Column 1& 2). The standard, conventional tonopen measures an average of 15.9 +/- 3.6 mmHg. The tonopen is typically used as a high IOP screening tool and thereby requires a high sensitivity. Here, the standard tonopen underestimates the higher specificity Goldmann and CATS IOP measurements in columns 4 & 5 at 17.9 +/- 3.1 mmHg and 17.0 +/-2.8 mmHg, respectively. The tonopen with a tip modified structured according to the idea of the invention measures a higher average: 18.3 +/- 2.9 mmHg. This is not statistically different that the results of the measurement produced with a more accurate Goldmann-type tonometer equipped with the CATS prism (p=0.08). The higher IOP and improved accuracy make the tonopen-type device with a tip structured according to the idea of the invention an improved tool with higher sensitivity and less variability.

TABLE 1

| CATS Tonopen | Column1 | Column2 | Column3 | Column4 | Column5 | Column6 |
|---|---|---|---|---|---|---|
| Eye | Tono tech | Tono flat | Tono CATS | CATS | GAT | CCT |
| 1 | 8 | 9 | 12 | 12 | 11 | 604 |
| 2 | 9 | 9 | 12 | 12 | 11 | 598 |
| 3 | 18 | 17 | 21 | 15 | 17 | 594 |
| 4 | 17 | 18 | 20 | 16 | 16 | 600 |
| 5 | 11 | 12 | 17 | 15 | 16 | |
| 6 | 12 | 13 | 20 | 13 | 14 | |
| 7 | 15 | 17 | 21 | 23 | 20 | 535 |
| 8 | 15 | 16 | 19 | 22 | 19 | 545 |
| 9 | 6 | 9 | 18 | 15 | 12 | 594 |
| 10 | 8 | 12 | 14 | 15 | 12 | 581 |
| 11 | 9 | 14 | 17 | 16 | 16 | |
| 12 | 8 | 15 | 17 | 15 | 15 | |
| 13 | 18 | 20 | 20 | 17 | 17 | 529 |
| 14 | 18 | 19 | 19 | 18 | 18 | 528 |
| 15 | 10 | 16 | 16 | 16 | 15 | 542 |
| 16 | 11 | 16 | 15 | 16 | 16 | 547 |
| 17 | 14 | 15 | 16 | 13 | 14 | 561 |
| 18 | 14 | 15 | 16 | 16 | 14 | 559 |
| 19 | 15 | 21 | 23 | 24 | 21 | 593 |
| 20 | 17 | 17 | 19 | 26 | 23 | 576 |
| 21 | 21 | 14 | 18 | 20 | 19 | 579 |
| 22 | 17 | 13 | 12 | 15 | 15 | 596 |
| 23 | 14 | 13 | 18 | 18 | 18 | 607 |
| 24 | 15 | 17 | 15 | 21 | 20 | 559 |
| 25 | 18 | 18 | 23 | 19 | 19 | 569 |
| 26 | 17 | 20 | 23 | 19 | 19 | 536 |
| 27 | 18 | 22 | 23 | 26 | 24 | 558 |
| 28 | 14 | 21 | 23 | 22 | 20 | 559 |
| 29 | 13 | 17 | 18 | 14 | 14 | 598 |
| 30 | 13 | 14 | 15 | 15 | 15 | 617 |
| 31 | 14 | 16 | 19 | 18 | 15 | 522 |
| 32 | 11 | 17 | 14 | 18 | 16 | 510 |
| 33 | 20 | 21 | 22 | 20 | 20 | 611 |
| 34 | 20 | 22 | 25 | 23 | 23 | 613 |
| 35 | 15 | 17 | 15 | 14 | 15 | 527 |
| 36 | 16 | 20 | 21 | 20 | 20 | 530 |
| 37 | 11 | 12 | 18 | 9 | 10 | 599 |
| 38 | 13 | 17 | 19 | 14 | 13 | 588 |
| 39 | 15 | 14 | 19 | 15 | 13 | 539 |
| 40 | 15 | 13 | 17 | 17 | 14 | 543 |
| 41 | 15 | 16 | 17 | 18 | 18 | 572 |
| 42 | 14 | 16 | 24 | 23 | 22 | 580 |
| 43 | 16 | 13 | 19 | 22 | 18 | 551 |
| 44 | 19 | 24 | 25 | 32 | 29 | 540 |
| 45 | 11 | 14 | 16 | 19 | 18 | 500 |
| 46 | 12 | 12 | 14 | 20 | 18 | 509 |
| Avg | 14.130435 | 15.934783 | 18.347826 | 17.956522 | 17 | |
| TD | 4.2498499 | 3.6140316 | 2.9424757 | 3.1167749 | 2.8139594 | |
| t-test | tech/phys | TonoCAT/flat | | | | |
| p= | 8.196E-05 | 6.547E-08 | 0.0848062 | 0.0083771 | 0.0005558 | |

FIG. 11 illustrates a retrofit of the existing pachymeter device with an embodiment of the invention. Specifically, FIG. 11 illustrates the situation in which the conventional tip 310 of the DGH ultrasound pachymeter of related art is equipped with an article of manufacture structured according to the embodiment 900 or the embodiment 950 of the invention. When applied to the cornea, the front surface of the so retrofitted transducer substantially conforms to the cornea thereby not distorting and/or thickening the cornea and avoiding the error of the measurement that results in a reading representing the cornea thicker than it is.

Conceptual proof of workability of an embodiment of the invention with an ultrasound pachymeter was provided by measuring the central corneal thickness (CCT) of eyes of 14 patients with the conventionally used OCT-based pachymetry (considered to be the “gold standard” in related art; results are presented in column 1 in the Table 2), with the conventional DGH ultrasound pachymeter equipped with the conventional probe 310 with a conventional tip 310A (results are presented in column 2 of Table 2), and with the DGH ultrasound pachymeter the tip of which has been modified according to the idea of the present invention (and, specifically, retrofitted to possess an inwardly-caved surface; results are presented in column 3 of Table 2, denoted as “CATS”). The results obtained with the use of the ultrasound pachymeter with a tip modified according to the idea of the invention repeatedly demonstrated values that are significantly closer to those obtained with the use of the PCT-based modality that those measured by conventional, not-modified DGH ultrasound instrument. The experimental results illustrated the difference with statistical significance p=0.0003.

TABLE 2

| CATS Pachymeter | Column1 | Column2 | Column3 |
|---|---|---|---|
| Sample (Eye) | CCT OCT | CCT DGH | CCT CATS |
| 1 | 587 | 590 | 590 |
| 2 | 589 | 601 | 595 |
| 3 | 548 | 571 | 560 |
| 4 | 550 | 563 | 560 |
| 5 | 555 | 584 | 564 |
| 6 | 552 | 576 | 571 |
| 7 | 563 | 589 | 575 |
| 8 | 560 | 583 | 570 |
| 9 | 538 | 556 | 546 |
| 10 | 550 | 560 | 560 |
| 11 | 493 | 505 | 498 |
| 12 | 493 | 501 | 489 |
| 13 | 497 | 537 | 509 |
| 14 | 502 | 529 | 513 |
| Avg | 541.21429 | 560.35714 | 550 |
| STD | 31.47148 | 30.382342 | 32.817678 |
| | T-CAT/DGH | | |
| p= | 0.0002517 | | |

### Protection Against Contamination..

At least one shortcoming of the use of any eye-contacting ophthalmological device is apparent from the very nature of its operation: such devices must touch the eye, and therefore, poses a risk of transferring various pathogens from one patient to another, or from patient to health care provider. While various methods have been utilized to reduce the transmission risk - including sterilization of the probe tip between examinations, the use of sterile gloves by the tester (to reduce a chance of re-contamination of a tip/probe portion of the device that may occur when reinserting the probe into the probe holder of the device), and wiping of the probe tip surface, to name just a few - these precautions were proven to be insufficient to reliably remove pathogens and contaminations. For these reasons, a more preferred method has been to fit the probe, or contact tip, of a contact ophthalmological device with a protecting cover during the measurement process, at least in order to prevent the detrimental transmission of pathogens. Such cover can be made disposable and is often formed from materials such as natural latex rubber or various hypoallergenic materials, as discussed, for example, in US 7,287,856 (which considers a typical cover that is dimensioned for use with a probe of a contact tonometer and structured to have a substantially and overall convex - as viewed from outside of the unfolded cover- area of the tip or closed end of the cover).

However, as a person of skill will readily appreciate upon the analysis of US 7,287,856 (the disclosure of which is incorporated herein by reference), the embodiment of the conventionally structured flexible cover discussed in this patent will necessarily substantially negate at least some if not all advantages (that would otherwise be provided by the curved surface of the tip/probe of the ophthalmological device) during the measurement of, for example, intraocular pressure because a space, gap, and/or air bubble would remain between the inner surface of the conventionally dimensioned cover and the cornea-contacting curved surface of the probe/tip of the embodiment of the invention. The presence of gap/space/bubble quite possibly would introduce additional yet unknown and/or non-correctable errors. Accordingly, the skilled person will now appreciate the novel structures of embodiments of a flexible cover for the transducer probe that are intended to be juxtaposed with such probes.

To this end, FIG. 12 is a cross-sectional view of an embodiment 1200 of a prophylactic cover for a tip of a probe structured according to FIG. 1A and/or FIG. 1B and/or FIG. 3. It is appreciated that embodiment 1200 is a thin rubber (and, therefore, flexible) sheath which, when not folded in any way as shown in FIG. 12, has a generally tubular shape with one end closed and the other end open. (In that, the embodiment 1200 in its unfolded form is substantially condom-like shaped, with the exception of the shape and structure of the very tip of the closed end, which is discussed below) The embodiment 1200 has a tip area 1201, at the closed end of the embodiment, characterized by a curved outer surface 1201A and a curved inner surface 1201B, and a wall 1202 connecting the tip areas 1201 with the open end 1204 (the wall 1202 forming the substantially cylindrical portion of the unfolded embodiment 1200). The wall 1202 may be complemented with a retention bead or ring bead 1203 circumferentially formed at the wall 1202 at the open end 1204 of the cover 1200. It is understood that, when the surface 1201A is viewed in a -z direction, the surface 1201A is perceived as a concave surface, and when the surface 1201B is viewed in a +z direction (that is, internally to the cover, through the open end 1204), the surface 1201B is perceived as a concave surface. The curved shapes of the surfaces 1201A and 1201B in at least one specific case can be made substantially identical with one another such that the thickness of the cover remains substantially constant at every point at least within the bounds of the tip area 1201. In a related implementation, at least the inner surface 1201B may curved as described above while the outer surface 1201A may be substantially flat.

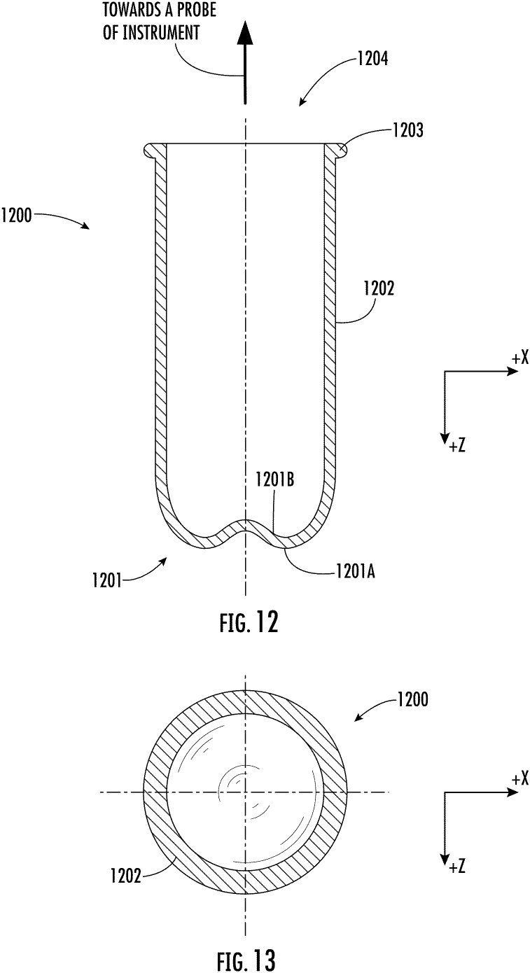

FIG. 13 schematically illustrates the cover 1200 as viewed from the open end 1204, in the +z direction, schematically showing the convex-like inner surface 1201B of the sip area 1201 and the optional circumferential retention bead or ring 1203. It is understood that, in a given embodiment of the prophylactic cover, at least the inner convex surface 1201B is preferably dimensioned to substantially conform to the shape of the front inwardly caved surface of the tip of the probe with which such cover is intended to be cooperated. (It is appreciated that when the target outer front surface of the tip of the probe is either substantially planar or even convex - as discussed, for example, in WO 2016/167827 - the inner surface shown as 1201B in FIGS. 12 and 13 is preferably dimensioned to be substantially congruent with such target outer front surface of the tip, according to the idea of the present invention, to avoid the introduction of unnecessary and unpredictable errors during the corresponding measurement of a parameter of the eye with such a probe covered with the cover.)

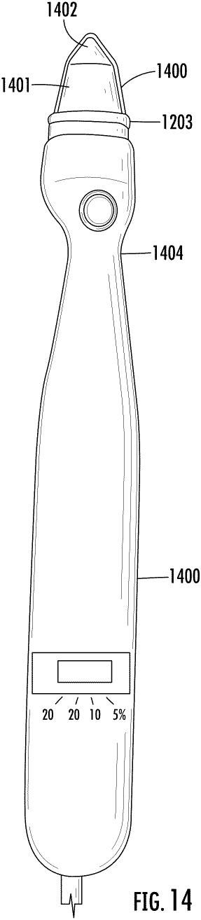

FIG. 14 shows a representative contact tonometer 1400 having a probe tip 1401 that is configured to have a spatially curved eye-contacting surface (for example, configured according to the embodiments shown in FIGS. 1A, 1B) and having a measurement part (tip of the probe) 1402 with an optionally disposable probe cover 1200 shown positioned over the probe 1401. The retention bead 1203 may be appropriately dimensioned to hold the probe tip cover 1200 onto the probe tip 1401. In one example, a contact tonometer 1400 is held by hand by medical personnel using the grip area 603 to perform the required test by contacting the contact front surface of the tip of the probe (which is covered by the tip cover 1200) to the eye (cornea) of the patient. The disposable tip cover 1200 protects the eye of the patient during the testing procedure.

While specific description chosen for the presented embodiments are recited, it is to be understood that, within the scope of the invention, the values of all of parameters may vary over wide ranges to suit different applications. Disclosed aspects, or portions of these aspects, may be combined in ways not listed above. For example, and in reference to FIGS. 8, 9A, 9B, 11, 11, a specific implementation of the cap/crown to the conventional transducer probe of a contact ophthalmological instrument may be configured to have a “skirt” circumferentially shielding at least a portion of the tip of the conventional probe with which such cap/crown is juxtaposed to form an embodiment of a hybrid transducer probe of the invention (see the outmost radially-protruding portion of the embodiment of FIG. 11). Additionally or in the alternative, the hollow 816 of substantially any embodiment of the cap/crown may contain a membrane of about 15 to 25 microns in thickness located across the hollow at the cornea-contacting end of it. The dimensions of the embodiment of FIGS. 8, 9A, 9B, 11, 11 are such that d is approximately 1.0 mm to about 1.5 mm, D is approximately 2.2 mm or so, and the outer diameter of the cap/crown at the cornea-contacting end of it is about 6.0 mm. (See, for example, FIG. 9 of U.S. Provisional Pat. Application No. 62/242,752.) Specifically, and intentionally, substantially any shape and dimension and/or combination of shapes and dimensions can be combined with one another to form a tonometer tip that is either a composite tonometer tip (the one incorporating a crown element dimensioned to modify a shape of a pre-existing tip prior to performing a tonometric measurement resulting in reduced measurement errors due to reduction of “buckling” of the cornea away from the tonometer sensor located in the central portion of the tip) or a single-piece and optionally monolithic tonometer tip possessing the outer surfaces shaped and/or dimensioned as discussed above to perform the measurement with so reduced measurement errors. Dimensions, if shown and/or discussed, are not limiting. Accordingly, the invention should not be viewed as being limited to the disclosed embodiment(s).

Whether or not a specific processor / microprocessor / electronic circuitry (that may be used to control the process of operation of the refocusing of the optical system as discussed) is shown in the Drawings, such microprocessor is controlled by instructions stored in a memory. The memory may be random access memory (RAM), read-only memory (ROM), flash memory or any other memory, or combination thereof, suitable for storing control software or other instructions and data. Those skilled in the art should also readily appreciate that instructions or programs defining the functions of the present invention may be delivered to a processor in many forms, including, but not limited to, information permanently stored on non-writable storage media (e.g. read-only memory devices within a computer, such as ROM, or devices readable by a computer I/O attachment, such as CD-ROM or DVD disks), information alterably stored on writable storage media (e.g. floppy disks, removable flash memory and hard drives) or information conveyed to a computer through communication media, including wired or wireless computer networks. In addition, while the invention may be embodied in software, the functions necessary to implement the invention may optionally or alternatively be embodied in part or in whole using firmware and/or hardware components, such as combinatorial logic, Application Specific Integrated Circuits (ASICs), Field-Programmable Gate Arrays (FPGAs) or other hardware or some combination of hardware, software and/or firmware components.

For the purposes of this disclosure and the appended claims, the expression of the type “element A and/or element B” has the meaning that covers embodiments having element A alone, element B alone, or elements A and B taken together and, as such, is intended to be equivalent to “at least one of element A and element B”.

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention. Within this specification, embodiments have been described in a way that enables a clear and concise specification to be written, but it is intended and will be appreciated that embodiments may be variously combined or separated without parting from the scope of the invention. In particular, it will be appreciated that all features described herein at applicable to all aspects of the invention.

When the present disclosure describes features of the invention with reference to corresponding drawings (in which like numbers represent the same or similar elements, wherever possible), the depicted structural elements are generally not to scale, and certain components are enlarged relative to the other components for purposes of emphasis and understanding. It is to be understood that no single drawing is intended to support a complete description of all features of the invention. In other words, a given drawing is generally descriptive of only some, and generally not all, features of the invention. A given drawing and an associated portion of the disclosure containing a description referencing such drawing do not, generally, contain all elements of a particular view or all features that can be presented is this view, at least for purposes of simplifying the given drawing and discussion, and directing the discussion to particular elements that are featured in this drawing. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this particular detail in the drawing may be implied unless the context of the description requires otherwise. In other instances, well known structures, details, materials, or operations may be not shown in a given drawing or described in detail to avoid obscuring aspects of an embodiment of the invention that are being discussed. Furthermore, the described single features, structures, or characteristics of the invention may be combined in any suitable manner in one or more further embodiments.

Moreover, if the schematic flow chart diagram is included, the depicted order and labeled steps of the logical flow are indicative of one embodiment of the presented method. Other steps and order of steps may be conceived that are equivalent in function, logic, or effect to one or more steps, or portions thereof, of the illustrated method. Without loss of generality, the order in which processing steps or particular methods occur may or may not strictly adhere to the order of the corresponding steps shown.

For the purposes of this disclosure and the appended claims, the use of the terms “substantially”, “approximately”, “about” and similar terms in reference to a descriptor of a value, element, property or characteristic at hand is intended to emphasize that the value, element, property, or characteristic referred to, while not necessarily being exactly as stated, would nevertheless be considered, for practical purposes, as stated by a person of skill in the art. These terms, as applied to a specified characteristic or quality descriptor means “mostly”, “mainly”, “considerably”, “by and large”, “essentially”, “to great or significant extent”, “largely but not necessarily wholly the same” such as to reasonably denote language of approximation and describe the specified characteristic or descriptor so that its scope would be understood by a person of ordinary skill in the art. The use of this term in describing a chosen characteristic or concept neither implies nor provides any basis for indefiniteness and for adding a numerical limitation to the specified characteristic or descriptor. As understood by a skilled artisan, the practical deviation of the exact value or characteristic of such value, element, or property from that stated may vary within a range defined by an experimental measurement error that is typical when using a measurement method accepted in the art for such purposes. As an example only, a reference to a vector or line or plane being substantially parallel to a reference line or plane is to be construed as such vector or line extending along a direction or axis that is the same as or very close to that of the reference line or plane (with angular deviations from the reference direction or axis that are considered to be practically typical in the art, for example between zero and fifteen degrees, more preferably between zero and ten degrees, even more preferably between zero and 5 degrees, and most preferably between zero and 2 degrees). A term “substantially flexible”, when used in reference to a housing or structural element providing mechanical support for a contraption in question, generally identifies the structural element the flexibility of which is higher than that of the contraption that such structural element is associated with. As another example, the use of the term “substantially flat” in reference to the specified surface implies that such surface may possess a degree of non-flatness and/or roughness that is sized and expressed as commonly understood by a skilled artisan in the specific situation at hand. For example, the terms “approximately” and about”, when used in reference to a numerical value, represent a range of plus or minus 20% with respect to the specified value, more preferably plus of minus 10%, even more preferably plus or minus 5%, most preferably plus or minus 2%.

The invention as recited in claims appended to this disclosure is intended to be assessed in light of the disclosure as a whole, including features disclosed in prior art to which reference is made.

What is claimed is:

1. A transducer probe comprising:
a probe body having:
a body axis,
a first portion of the probe body, the first portion defined by a front surface of a transducer of said contact ophthalmological instrument,
a second portion of the probe body circumscribing and forming a ridge above the first portion of the probe body,
wherein the transducer probe is configured for use with the contact ophthalmological instrument employing said transducer, and
wherein a front surface of the probe body is transverse to the body axis and is inwardly shaped.

2. A transducer probe according to claim 1, wherein the front surface of the transducer is substantially circumscribed with material different from a material of the front surface of the transducer and/or the front surface of the probe body is substantially opaque to light.

3. A transducer probe according to claim 1, wherein:
the front transducer surface defines at least an axial portion of the front surface of the probe body; and/or
the front surface of the transducer is substantially opaque to light.

4. A transducer probe according to claim 3, wherein:
the front surface of the transducer is substantially circumscribed with a
layer of material different from a material of the front surface of the transducer; and/or
the front surface of the transducer is separated from the second portion of the probe body, in a radial direction, with said layer of material.

5. A transducer probe according to claim 1, wherein:
a cornea-contacting surface of the second portion of the probe body a first non-zero surface curvature with a first sign in a plane parallel to the body axis, and/or
the cornea-contacting surface of the second portion of the probe body is dimensioned to be substantially tangentially-parallel with the front surface of the transducer along a perimeter of said front surface of the transducer.

6. A transducer probe according to claim 5, wherein the front surface of th4e transducer is substantially flat .

7-9. (canceled)

10. A transducer probe according to one claim 1, wherein said probe body is substantially monolithic .

11. A transducer probe according to claim 1, wherein
(11A) the transducer includes a piezo or pressure sensor element; and/or
(11B) the front surface of the transducer has a non-zero surface curvature with a first sign; and/or
(11C) a surface of the second portion of the probe body and the front surface of the transducer are dimensioned to be tangentially parallel substantially at every point of a perimeter of the front surface of the transducer.

12. A transducer probe according to claim 11, wherein
a sign of non-zero curvature of a cornea-contacting surface of the second portion of the probe body is equal to a sign of non-zero curvature of the front surface of the transducer.

13. A transducer probe according to claim 5, wherein the first sign is equal to a sign of a curvature of a surface of the cornea.

14. A transducer probe according to claim 1, wherein front surface of the probe body surface is axially-symmetric about said body axis and/or the front surface of the transducer defines a portion of a spherical surface.

15. (canceled)

16. A transducer probe according to claim 1, wherein a front surface of said ridge has a surface curvature with a sign that is opposite to a sign of a surface curvature of a cornea-contacting surface of the second portion of the probe body, the front surface of said ridge being substantially co-axial with the front transducer surface.

17. A transducer probe according to claim 1, wherein the front surface of the transducer surface is substantially opaque to light and the transducer probe is configured as a tonometer probe for use with a hand-held portable contact tonometer.

18. (canceled)

19. A method for measuring an intraocular pressure (IOP) of an eye, the method comprising:
equipping a hand-held contact ophthalmological instrument with a transducer probe according to claim 1,
mechanically connecting at least the front surface of the transducer with the cornea of the eye; and
operating the transducer to take a measurement of the IOP without transmitting light through a front surface of the transducer and/or through the first portion of a probe body and/or through the second portion of the probe body towards the cornea.

20. (canceled)

22. A method according to claim 19, further comprising:
forming a barrier to transfer of microorganisms between the eye and the probe body prior to said mechanically connecting, and wherein said mechanically connecting includes mechanically connecting the at least the front surface of the transducer with the cornea through said barrier.

23. A method according to claim 22, wherein said barrier is dimensioned to have a surface that substantially conforms to a corneal surface of the eye.

24-26. (canceled)

27. A method according to claim 19, wherein said operating includes repositioning the front surface of the transducer with respect to the cornea.

28. A method according to claim 19, wherein said operating includes repositioning both the first portion of the probe body and the second portion of the probe body with respect to the cornea.

29. (canceled)

30. A method according to claim 19, wherein:
(30A) said mechanically connecting includes establishing a direct physical contact between the front surface of the transducer and the cornea; or
(30B) said mechanically connecting includes mechanically connecting the at least the front surface of the transducer with the cornea through a thin-film of elastic material disposed therebetween.

31. A method according to claim 19, wherein said equipping includes equipping a hand-held contact tonometer with the transducer probe having a front surface substantially opaque to light.

32-91. (canceled)
